(19) United States
(12) Patent Application Publication
(10) Pub. No.: US 2023/0023605 A1
(43) Pub. Date: Jan. 26, 2023

(54) Visual Field Testing Apparatus

(71) Applicant: FINDEX Inc., Tokyo (JP)

(72) Inventor: Teruo AIBARA, Tokyo (JP)

(21) Appl. No.: 17/937,780

(22) Filed: Oct. 4, 2022

Related U.S. Application Data
(63) Continuation-in-part of application No. PCT/JP2021/016860, filed on Apr. 27, 2021.

(30) Foreign Application Priority Data
Jun. 16, 2020 (JP) 2020-103472

(51) Int. Cl. A61B 3/024; A61B 3/00

(52) U.S. Cl. CPC A61B 3/024; A61B 3/0091; A61B 3/005

(57) ABSTRACT

A visual field testing apparatus may comprise a display; a line of sight detection unit that detects a line of sight of a user; a visual recognition determination unit that carries out determination as to whether or not the user has visually recognized the target; a target display unit that causes a gazing target and a measurement target to be sequentially displayed at the display; and a visual field testing unit that carries out visual field testing by causing visual recognition determination to be carried out with respect to the measurement target by the visual recognition determination unit as the gazing target and the measurement target are sequentially displayed by the target display unit. The visual field testing unit may have a central scotoma possessor testing mode in which the gazing target is displayed at the display 13 for the eye opposite the eye being tested.

### CROSS-REFERENCE TO RELATED APPLICATION, BENEFIT/PRIORITY CLAIM(S), AND INCORPORATION BY REFERENCE

This application is a continuation-in-part of and claims benefit under 35 USC 120 and 365(c) to copending International Application No PCT/JP2021/016860, entitled “Visual Field Testing Apparatus”, filed 27 Apr. 2021; and further claims benefit of priority under 35 USC 119(a)-(d) to Japanese Patent Application No 2020-103472, entitled “Visual Field Testing Apparatus”, filed 16 Jun. 2020, the contents of both of which applications are incorporated herein in their entireties by reference.

### FIELD OF THE INVENTION

The present invention relates to a visual field testing apparatus.

### BACKGROUND

In a visual field testing apparatus in accordance with one embodiment, a user might, for example, be made to use a button or other such input apparatus to input whether or not a target has been seen by his or her eye and recognized.

However, depending on the user, there have been cases with such an embodiment in which the user is unfamiliar with operation of the input apparatus, or due to tension or the like is unable to skillfully operate the input apparatus, and so fails to properly carry out input therewith, as a result of which there has been inability to accurately carry out visual field testing.

In contradistinction thereto, with a visual field testing apparatus in accordance with another embodiment, a line of sight which is the direction in which a user is looking might be automatically detected by a line of sight detection apparatus, and determination might be automatically made as to whether or not the user visually recognizes the target.

In accordance with such an embodiment, during visual field testing, two targets— these being a gazing target for causing the line of sight to return to the center of the visual field where gazing (ocular fixation) is made to take place, and a measurement target for measuring visual field—might be alternately used. Where this is the case, there may be occurrence of situations in which a user who has a scotoma affecting the vicinity of the center of the visual field may be unable to visually recognize the gazing target and/or be unable to carry out visual field testing.

There is therefore a need to provide a visual field testing apparatus such as will permit visual field testing to be carried out even on a user who has a scotoma affecting the vicinity of the center of the visual field.

### SUMMARY OF INVENTION

A visual field testing apparatus associated with one embodiment of the present invention, in the context of a visual field testing apparatus for testing a range of visual field of a user, may comprise a display for displaying a target.

In accordance with some embodiments, the visual field testing apparatus may further comprise a line of sight detection unit that detects a line of sight of the user and that outputs line of sight information pertaining to a direction of the line of sight.

In accordance with some embodiments, the visual field testing apparatus may further comprise a visual recognition determination unit that carries out determination as to whether or not the user has visually recognized the target based on the line of sight information and position information of the target.

In accordance with some embodiments, the visual field testing apparatus may further comprise a target display unit that causes the target in a form of a set consisting of a gazing target and a measurement target to be sequentially displayed at the display.

In accordance with some embodiments, the visual field testing apparatus may further comprise a visual field testing unit that carries out visual field testing by causing visual recognition determination to be carried out with respect to the measurement target by the target visual recognition determination unit as the gazing target and the measurement target are sequentially displayed by the target display unit.

In accordance with some embodiments, the visual field testing apparatus may be such that the display has a display for a right eye and a display for a left eye.

In accordance with some embodiments, the visual field testing apparatus may be such that the visual field testing unit has a central scotoma possessor testing mode in which the target display unit is controlled so as to cause the gazing target to be displayed by whichever of the display for the right eye or the display for the left eye is opposite an eye being tested.

In accordance with such an embodiment of the present invention, a visual field testing unit may have a central scotoma possessor mode, and visual field testing may be properly carried out even on a user who has a scotoma affecting the vicinity of the center of the visual field.

Other embodiments, systems, methods, and features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram showing in simplified fashion the constitution of a visual field testing apparatus associated with an embodiment of the present invention.

FIG. 2 is a sectional view of the principal components in, and showing in simplified fashion the constitution of, an HMD associated with an embodiment of the present invention.

FIG. 3 is a perspective view of the principal components in, and showing in simplified fashion the constitution of, an HMD associated with an embodiment of the present invention.

FIG. 4 is a drawing showing locations at which measurement targets associated with an embodiment of the present invention might be displayed.

FIG. 5 is a drawing showing examples of output screens displaying results of visual field testing associated with an embodiment of the present invention.

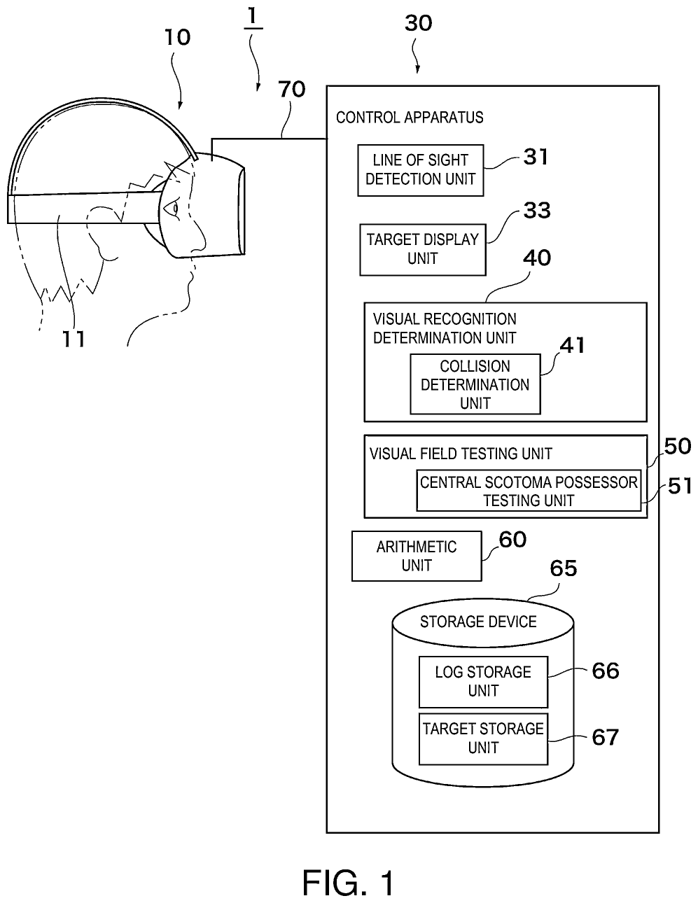

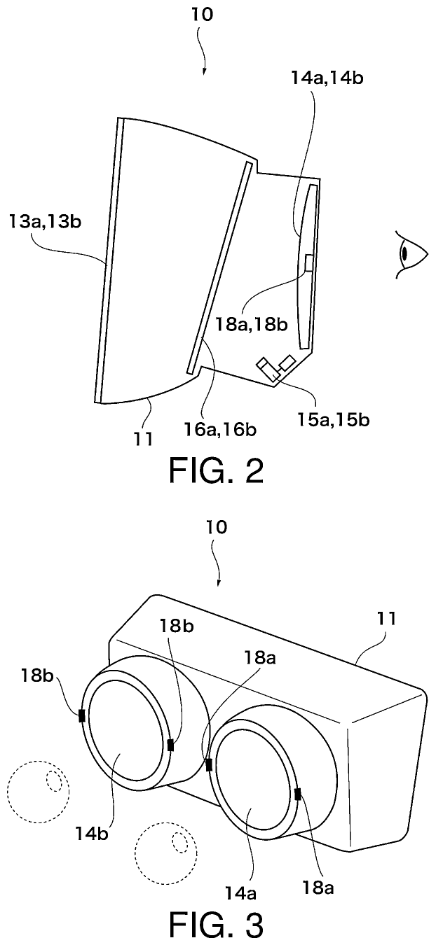

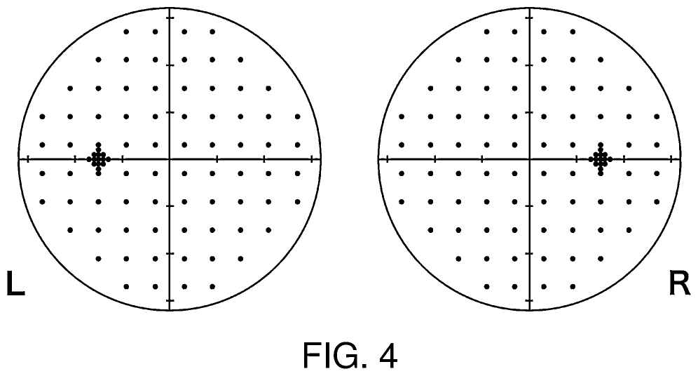

### EMBODIMENTS FOR CARRYING OUT INVENTION

Visual field testing apparatus 1, which is an embodiment of the present invention, will be described in detail hereinbelow with reference to the drawings. In accordance with the present embodiment, visual field testing apparatus 1 carries out visual field testing while automatically detecting a line of sight which is a direction in which the user is looking with his or her eye(s) and causing a determination to be made as to whether a target which is a visual symbol that is displayed at a display apparatus has been seen by the eye(s) of the user and recognized, i.e., whether or not it has been visually recognized.

Visual field testing apparatus 1 in accordance with the present embodiment may comprise head mounted display (HMD) 10, control apparatus 30, and communication cable 70 which connects HMD 10 and control apparatus 30.

HMD 10 may comprise case 11 which might include a belt for mounting thereof on the head of a user who is a test subject, display 13, convex lens 14, camera 15, hot mirror 16, and/or near-infrared emitter 18, and might be equipped with line of sight detection functionality for cooperating with line of sight detection unit 31, described below, to automatically detect a line of sight which is a direction in which the user on whom HMD 10 is mounted is looking with his or her eye(s).

Display 13 might be a liquid crystal display in which display 13a for the right eye of the user and display 13b for the left eye thereof are installed so as to respectively face the fronts of his or her left and right eyes. Respectively installed to the right and left between display 13 and the eyes of the user there may be a convex lens 14a for the right eye and a convex lens 14b for the left eye. Where this is the case, an image displayed at display 13 might be made to appear at the eye(s) of the user by way of convex lens 14.

Except where otherwise clear from context, note that use of the singular or plural herein should not be understood in a limiting or exclusionary sense where this would constitute an unreasonable interpretation of the present invention. For example, depending on the embodiment in question, it may be that both eyes are tested during a round of testing but only one eye is actively undergoing testing at any given moment. Likewise, depending on the embodiment in question, it may be that multiple sets of gazing targets and measurement targets are displayed during a round of testing but only one set, and more particularly only either the gazing target or the measurement target of that set, is actively being displayed at any given moment. Furthermore, depending on the embodiment in question, it may be that both eyes are tested but only one eye is being described by way of example. Moreover, as noted below, independent displays 13a, 13b may, for example, be used for the right eye and the left eye, or a single large display 13 that has been divided into a region 13a for the right eye and a region 13b for the left eye may be used, and depending on the embodiment in question the various units and other components for testing of the right eye and the left eye may similarly take the form of a single component for both eyes or separate components for each of the respective eyes. It should therefore be noted that regardless of whether the present specification makes reference in the singular to display 13, lens 14, camera 15, mirror 16, emitter 18, and so forth, or conversely makes reference in the plural to displays 13a, 13b, lenses 14a, 14b, cameras 15a, 15b, mirrors 16a, 16b, emitters 18a, 18b, and so forth, such references should except where otherwise clear from context be understood in a collective or nonexclusionary sense, without limitation as to whether there is a single such component for both eyes or separate components for the respective eyes. Similarly, references made herein to gazing target(s), measurement target(s), and the various apparatus(es) and unit(s) and so forth should, except where otherwise clear from context, be understood as nonlimiting with respect to number.

Camera 15 might be a near-infrared camera for capturing images of the eyes of the user, video capture of the left and right eyes of the user being carried out thereby through use of near-infrared light which is nonvisible light. Camera 15a for the right eye and camera 15b for the left eye might likewise be installed at camera 15.

Respectively installed to the right and left between display 13 and convex lens 14 there may be hot mirrors 16a, 16b having multilayer films which reflect near-infrared light and allow visible light to pass therethrough. Where this is the case, visible light of an image irradiated from display 13 might be made to pass through hot mirror 16, and nonvisible light of near-infrared light irradiated from emitter 18 might be reflected by hot mirror 16.

Emitter 18 might be an LED (IR-LED) that causes near-infrared light to be irradiated as illumination for capturing images of the eyes of the user. Emitters 18a, 18b may be respectively installed to the left and right so as to face the left and right eyes of the user at the periphery of convex lens 14.

Camera 15 may be installed at the side toward the eyes, which is the side opposite display 13, from hot mirror 16. Near-infrared light which directly irradiates the eyes of the user from emitter 18 might be reflected by the eyes of the user and thereafter be made to pass through convex lens 14 and be reflected by hot mirror 16 to arrive at camera 15 where an image might be captured therefrom.

Control apparatus 30 may comprise central processing unit(s) (CPU) or other such arithmetic unit(s) 60 for carrying out various types of operations, as well as hard disc drive(s) (HDD) for storing various types of information, and/or random access memory or memories (RAM) capable of being used as a work area during arithmetic processing, or other such storage device(s) 65.

Storage device 65 might comprise line of sight log storage unit 66 which records line of sight information detected by line of sight detection unit 31, described below; and might further comprise target storage unit 67 which records target information to be displayed at display 13.

Furthermore, control apparatus 30 might comprise—in terms of functionalities—line of sight detection unit 31, target display unit 33, visual recognition determination unit 40, and/or visual field testing unit 50, these functionalities being implemented by causing prescribed program(s) stored at storage device 65 to be executed by arithmetic unit 60, for example.

Line of sight detection unit 31 might detect the direction in which the user is looking, i.e., the line of sight, based on captured image(s) of the eye(s) of the user as output by camera 15. More specifically, line of sight detection unit 31 might output line of sight information indicating the direction of the line of sight in the form of two angular components (θ, ϕ) which are polar coordinates (spherical coordinates) as line of sight information (line of sight angle).

Establishment of the polar coordinate system may be carried out during a calibration operation which might be carried out prior to the start of visual field measurement. More specifically, the user might be made to stare at a previously designated target and the image thereof might be made to correspond to the polar coordinate system origin (center of eyeball), following which an image at which the user is made to stare at a point representing a different designated set of polar coordinates might be stored. By determining the correlation between the locations of the centers of the pupils captured in the respective images and the respective polar coordinates at which the user was made to stare, it will be possible to determine the line of sight angle of the polar coordinates from the images.

This line of sight detection may be carried out independently for the right eye and the left eye, line of sight information being recorded as a function of time at line of sight log storage unit 66 at intervals of approximately 15 ms, for example. Included within the line of sight information which is recorded at line of sight log storage unit 66 there may be the aforementioned two angular components (θ, ϕ) which are polar coordinates (spherical coordinates).

Target display unit 33 may cause targets of prescribed size(s) to be sequentially displayed at prescribed locations(s) at display 13 based on target information recorded at target storage unit 67. Here, in accordance with the present embodiment, targets in the form of two targets—these being a gazing target for causing the line of sight to return to a location in the vicinity of the center of the visual field where gazing (ocular fixation) is made to take place, and a measurement target for measuring visual field—might be used, and a target set which consists of a measurement target and a gazing target might be recorded at target storage unit 67.

Note that location information indicating display locations of respective targets in the form of sets of the aforesaid two angular components (θ, ϕ) which are polar coordinates may be used as the target information which is stored at target storage unit 67. These two angular components would in such case define a target angle indicating a target location expressed in angular form.

FIG. 4 shows display locations of measurement targets in accordance with one example. In accordance with the present embodiment, to carry out measurement of visual field within a visual field angle of 30°, for example, measurement targets might be displayed at points at 76 locations arranged after the fashion of the intersections of a grid, and at points at 13 locations arranged in concentrated fashion in the vicinity of the blind spot, for a total of 89 locations (Humphrey visual field test; center 30-2). The visual field testing method may of course be varied as appropriate, and the locations at which the targets are displayed at display 13 and so forth may be varied as appropriate in correspondence to the visual field testing method employed.

Note that in accordance with the present embodiment not all measurement targets are displayed at the coordinate locations shown in FIG. 4, display locations being adjusted as appropriate so as to cause all measurement targets to be displayed within a prescribed visual field angle (18° in the present embodiment) at display 13. Such adjustment may be carried out in advance prior to measurement, the post-adjustment locations being recorded in the form of target information at target storage unit 67. Furthermore, in accordance with the present embodiment, the gazing target is not stationary with respect to the center of display 13 but is displayed at a location which has been made to agree with the adjustment of the measurement target (the location for which the visual field angle of the post-adjustment measurement target is the same as was the case prior to adjustment).

The reason for this is, because measurement targets are displayed at locations which are removed by some distances from the center of display 13, and because the larger the line of sight angle of the line of sight with respect to the screen at display 13 the lower will be the precision with which the line of sight will be detected by line of sight detection unit 31, to cause the measurement targets to be displayed so as to be located within a prescribed visual field angle (18° in the present embodiment) from the center of the optical system at display 13.

More specifically, if a measurement target would be at a location outside the range of a visual field angle of 18° from the center of the optical system at display 13, adjustment might in accordance with the present embodiment be carried out so as to cause said measurement target to be moved in translational fashion to a location where it will be displayed within 18° at display 13. At such time, so that there is no change in the angle by which the line of sight moves in going from the immediately prior gazing target in that same set to said measurement target, the immediately prior gazing target is likewise made to move in translational fashion by the same distance and in the same direction as said measurement target.

By thus causing measurement targets to be displayed at locations within a visual field angle of 18° at display 13, it will be possible to detect the line of sight in stable and highly precise fashion without experiencing decrease in the precision of detection thereof by line of sight detection unit 31.

Visual recognition determination unit 40 which comprises collision determination unit 41 may carry out determination as to whether or not a target has been visually recognized by a user based on a line of sight detected by line of sight detection unit 31 and the coordinates of a measurement target displayed at display 13.

Collision determination unit 41 may carry out determination as to whether or not the target has been visually recognized by the user based on whether or not an imaginary line that is an extension of a line of sight vector direction detected by line of sight detection unit 31 within a prescribed coordinate system would physically collide with the target in question.

In accordance with the present embodiment, a determination might be made that there has been a collision and that visual recognition has taken place if upon comparison of the line of sight angle detected by line of sight detection unit 31 and the target angle indicating the location of the target in question, the angular difference therebetween is within 2° within the same polar coordinate system.

Visual field testing unit 50 may control target display unit 33 and visual recognition determination unit 40, and may cause visual field testing of the user to be carried out by sequentially causing determination with respect to visual recognition of respective targets to be carried out by visual recognition determination unit 40 while causing sets of gazing targets and measurement targets to be sequentially displayed at prescribed locations by target display unit 33.

One embodiment of the present invention may employ a normal mode and a central scotoma possessor mode, where the central scotoma possessor mode is for carrying out visual field testing of a central scotoma possessing user who has a scotoma affecting the vicinity of the center of the visual field of the left and/or right eye, and where the normal mode is for carrying out visual field testing of a user who does not have such a central scotoma.

When in normal mode, to carry out visual field testing of the right eye, target display unit 33 might cause targets to be displayed at only display 13a for the right eye; and to carry out visual field testing of the left eye, target display unit 33 might cause targets to be displayed at only display 13b for the left eye.

Visual field testing unit 50 may comprise central scotoma possessor testing unit 51. Central scotoma possessor testing unit 51 may perform visual field testing in central scotoma possessor mode for carrying out visual field testing of a central scotoma possessing user who has a scotoma affecting the vicinity of the center of the visual field of the left and/or right eye.

Because the gazing target for causing the line of sight to return to a location in the vicinity of the center of the visual field is displayed in the vicinity of the center of the visual field, it may be the case that a central scotoma possessing user will be unable to visually recognize the gazing target, as a result of which it will be impossible to carry out testing through use of the visual field testing in normal mode.

To address this, when in central scotoma possessor mode, central scotoma possessor testing unit 51 might cause the gazing target—which is part of the set of targets consisting of the gazing target and the measurement target—to be displayed each time at the display of display 13 which is for the eye opposite the eye being tested.

For example, when carrying out visual field testing of the right eye of a user who has a scotoma affecting the vicinity of the center of the visual field of the right eye, control might be carried out by central scotoma possessor testing unit 51 so as to cause target display unit 33 to display all gazing targets at display 13b for the left eye, and display all measurement targets at display 13a for the right eye.

Based on the knowledge that the left and right eyes of a human being are ordinarily such that the lines of sight of the left and right eyes are approximately parallel, if the gazing target is displayed at the display of display 13 which is for the eye opposite the eye being tested, causing visual recognition of the gazing target to be carried out by this opposite eye will allow the line of sight of the eye being tested to also be made to return to the center of the visual field. It is therefore possible for visual field testing of a central scotoma possessing user to be carried out not in normal mode but as a result of causing a central scotoma possessor mode to be provided by central scotoma possessor testing unit 51.

Moreover, in accordance with the present embodiment, when the gazing target is displayed at the display of display 13 which is for the opposite eye, central scotoma possessor testing unit 51 might carry out control of target display unit 33 so as to cause brightness to be increased to a value greater than would be employed in normal mode, and/or so as to cause the gazing target to be displayed larger than would be employed in normal mode.

More specifically, whereas a target might be displayed by display 13 when in normal mode as a circle 4 mm² in size and/or so as to have a brightness of 413 cd/m², the gazing target when in central scotoma possessor testing mode might be displayed so as to be 16 mm² in size and/or so as to have a brightness of 826 cd/m², for a brightness ratio between modes of 2×, and/or an area ratio between modes of 4× (radius ratio of 2×). Note that while the sizes of the targets given here may be understood to be expressed in terms of the sizes thereof when displayed on a screen which is, for example, 30 cm in front of the eye, these values are presented by way of example and it should be understood that there is no limitation with respect to the distance of the screen from the eye, or with respect to target size or target brightness, or the ratios thereof between modes.

Because there is a possibility that a user who has a central scotoma at one eye will have decreased sensitivity at the opposite eye, by thus causing a gazing target which is brighter and/or larger than when in normal mode to be displayed at the opposite eye when in central scotoma possessor testing mode, it will be possible to more properly carry out visual field testing.

It is of course possible to adjust as appropriate the brightness and/or size of the gazing target which is displayed at the eye opposite the eye being tested, and while either or both of these may be made to be the same as when in normal mode, based upon consideration of the decrease in sensitivity of the eyes of central scotoma possessors, the reliability of testing, and so forth, it is preferred that the brightness and size of the gazing target should each be 1.5× to 3× that which is employed when in normal mode.

The foregoing being description of the constitution of visual field testing apparatus 1 in accordance with one embodiment, description will next be given with regard to an exemplary visual field testing method that employs visual field testing apparatus 1 in accordance with the foregoing embodiment.

Note that visual field testing apparatus 1 may perform visual field testing by executing visual field testing program(s) stored at storage device 65.

Description will first be given with respect to visual field testing such as might, for example, be carried out when in normal mode.

During visual field testing, HMD 10 might be placed on the head of a user who is a test subject, and an instruction might be given to the test subject to the effect that “targets will be sequentially displayed at display 13, so please keep looking at the targets”. In carrying out measurement, calibration might first be carried out, at which time adjustment might be carried out so that line of sight detection unit 31 can accurately detect the line of sight of the test subject.

Following initial adjustment, target display unit 33 might cause targets to be displayed at display 13. Target display unit 33 might cause targets for testing of the right eye and targets for testing of the left eye to both be displayed in random and sequential fashion at display 13 based on target information recorded at target storage unit 67. That is, in accordance with the present embodiment, visual field testing of the right eye and visual field testing of the left eye might be carried out in a single round of testing.

Note that where targets for the left eye are displayed only at display 13b for the left eye, and targets for the right eye are displayed only at display 13a for the right eye, it will be the case that only a target for either the right eye or the left eye will be displayed at display 13a for the right eye or display 13b for the left eye at any given time during testing.

As described above, a gazing target and a measurement target might be recorded as a target set, and target display unit 33, before causing display of a measurement target, might cause display of the gazing target which is part of the same set as that measurement target, and thereafter cause display of that measurement target. That is, target display unit 33 might cause gazing targets and measurement targets to be displayed at display 13 in alternating fashion.

Switching of targets may be such that when a determination is made by visual recognition determination unit 40 that a target which is being displayed has been visually recognized, target display unit 33 might cause switching to the next target to occur and cause this to be displayed. Furthermore, following display of a target, if visual recognition does not occur despite passage of 2.5 seconds, this might be taken to be a timeout condition, a determination might be made that visual recognition did not occur, and the next target set might be displayed. Where a target that resulted in generation of a timeout condition is a measurement target, said measurement target might be scheduled for remeasurement. Note, however, that depending on the conditions under which each of the various test patterns are terminated, there may nonetheless be cases in which a measurement target that had been scheduled for remeasurement is not remeasured.

As targets are sequentially displayed at display 13 by target display unit 33 in this fashion, the line of sight of the test subject might be detected by line of sight detection unit 31, and visual recognition determination unit 40 might determine whether or not recognition by the test subject has taken place for each target based on the line of sight information which is the output of line of sight detection unit 31.

Visual recognition determination unit 40 may be such that gazing targets and measurement targets which are displayed in sequential and alternating fashion are subjected to visual recognition determination which is sequentially carried out by collision determination unit 41 based on line of sight information which is sequentially output from line of sight detection unit 31 following display of respective targets. In the event that a determination is made by collision determination unit 41 that a target has not been recognized and said target is a measurement target, visual recognition determination unit 40 may cause said measurement target to be scheduled for remeasurement.

Note, however, that depending on the conditions under which each of the various test patterns are terminated, there may be cases in which a measurement target that had been scheduled for remeasurement is not remeasured. When one pass of visual recognition determination has been carried out on all measurement targets, this may then be followed by visual recognition determination which is again carried out by visual recognition determination unit 40 on any measurement target(s) that were scheduled for remeasurement.

Target sets scheduled for remeasurement may also be displayed in random and sequential fashion at display 13, and visual recognition determination may be carried out with respect thereto. Where a measurement target cannot be determined to have been visually recognized despite remeasurement thereof, a decision might be rendered by visual field testing unit 50 in accordance with the present embodiment that there is a scotoma thereat; and where a measurement target is determined to have been visually recognized upon remeasurement thereof, this might in accordance with the present embodiment be scheduled to be measured yet again. Where a measurement target is also determined to have been visually recognized the third time that it is measured, a decision might be rendered by visual field testing unit 50 in accordance with the present embodiment that there is no scotoma thereat; where a measurement target is determined not to have been visually recognized when measured for the third time, a decision might be rendered by visual field testing unit 50 in accordance with the present embodiment that there is a scotoma thereat.

Moreover, where a determination is made by collision determination unit 41 that visual recognition has occurred, the time from display of said measurement target until visual recognition thereof by the test subject, i.e., the time from display of the measurement target until detection of the line of sight that collided therewith, might be recorded as the response time at storage device 65.

Where this response time is, for example, not greater than 100 ms, because this might be deemed to be too short a response time, and it is conceivable that the reason for such a short response time could be that a subsequent target just happened to have been acquired in the instant that the target was switched, or that where a previous target and a subsequent target are close this created a situation in which the line of sight and the target just happened to collide in the instant that the target was switched, collision determination unit 41 might in accordance with the present embodiment treat this as an invalid visual recognition determination, and said measurement target might be scheduled for remeasurement.

Furthermore, in the event that the response time is a prescribed time or longer, collision determination unit 41 might treat this as a situation in which too much time has been taken for response, and might cause said measurement target to be scheduled for remeasurement. In accordance with the present embodiment, note that it is only measurement targets that are scheduled for remeasurement.

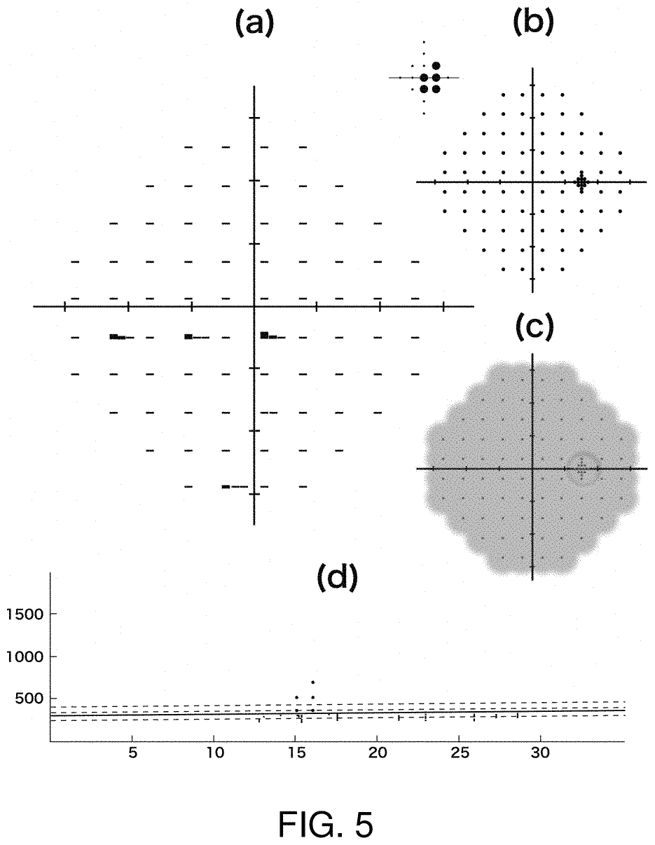

When visual recognition determination of all target sets—including those scheduled for remeasurement—as described above has been completed, visual field testing unit 50 might output the results of visual field testing to a prescribed display apparatus (not shown) based on the results of visual recognition carried out by visual recognition determination unit 40. FIG. 5 shows examples of output screens displaying exemplary results of visual field testing.

At (a) in FIG. 5, heights indicate response times at respective measurement targets exclusive of the region of the blind spot, multiple measurement results for targets subject to remeasurement being shown in horizontally clustered fashion. At (b) in FIG. 5, locations of measurement targets at which decisions were rendered that there was a scotoma thereat are shown, measurement targets at which decisions were rendered that there was a scotoma thereat being indicated by large circles.

At (c) in FIG. 5, respective measurement targets together with both the response times thereat as well as the locations at which decisions were rendered that there was a scotoma thereat are indicated by color and density, regions in the vicinity of targets at which response time was short being shown in light green, the shade of green shown being darker the longer the response time thereat, with regions in the vicinity of scotomas being shown in red. At (d) in FIG. 5, measurement target response time is shown as a function of visual field angle, the horizontal axis being visual field angle, and the vertical axis being response time.

The foregoing being description with respect to visual field testing when in normal mode, description will next be given with respect to a method for visual field testing when in central scotoma possessor mode such as may be carried out by central scotoma possessor testing unit 51, description being primarily given with respect to the portions thereof that are different from normal mode. When in central scotoma possessor mode which is for visual field testing of a user who has a central scotoma at one eye, the method for displaying gazing targets in accordance with the present embodiment is different from that employed in the foregoing normal mode.

Following initial adjustment, in causing targets to be displayed at display 13 by target display unit 33, during testing of the eye at which the user has a central scotoma, central scotoma possessor testing unit 51 of visual field testing unit 50 might control target display unit 33 so as to cause all gazing targets to be displayed at the display of display 13 which is for the eye opposite the eye being tested.

As a result, because in carrying out visual field testing, for example, of the right eye of a user who has a central scotoma at the right eye, all gazing targets would be displayed at display 13b for the left eye, by using the left eye to visually recognize the gazing target it will be possible to cause the line of sight of the right eye to be made to return to the vicinity of the center of the visual field, and it will be possible to properly carry out visual field testing using measurement targets displayed at display 13a for the right eye. Of course, in accordance with the present example, in carrying out visual field testing of the left eye at which there is no central scotoma, the gazing targets and the measurement targets would both be displayed at display 13b for the left eye in similar fashion as when in normal mode.

As targets are sequentially displayed at display 13 by target display unit 33 in this fashion, the line of sight of the test subject might be detected by line of sight detection unit 31, and visual recognition determination unit 40 might determine whether or not recognition by the test subject has taken place for each target based on the line of sight information which is the output of line of sight detection unit 31, as a result of which visual field testing unit 50 might cause the results of visual field testing to be output based on the results of visual recognition carried out by visual recognition determination unit 40 in similar fashion as when in the foregoing normal mode.

Above, description has been given with respect to visual field testing apparatus 1 associated with the present embodiment; in accordance with the present embodiment, where visual field testing unit 50 comprises central scotoma possessor testing unit 51, it will be possible by causing operations to be carried out in central scotoma possessor mode to properly carry out visual field testing of a user who has a central scotoma at only one eye.

While embodiments of the present invention have been described above, embodiments for carrying out the present invention are not limited to the foregoing embodiments, a great many variations being possible without departing from the gist of the present invention. For example, whereas the foregoing embodiment employed a noncontact-type line of sight detection unit in which the point on which the eye is fixated is estimated from the pattern of near-infrared light that is reflected from the cornea, so long as it permits detection of the line of sight of the user, it is possible to employ a contact-type line of sight detection unit or other such appropriate type of line of sight detection unit.

Furthermore, whereas in the foregoing embodiment the visual recognition determination unit carried out determination as to whether or not the user had visually recognized the target based on a determination made by a collision determination unit as to whether or not an imaginary line that is an extension of the direction of a line of sight detected by a line of sight detection unit would physically collide with the target in question, it is also possible to cause visual recognition determination to be carried out using logical determinative techniques that make use of logged line of sight information.

Furthermore, whereas the foregoing embodiment employed a polar coordinate system for lines of sight and line of sight position information, it is also possible to employ a three-dimensional rectangular coordinate system therefor. Furthermore, whereas in the foregoing embodiment independent displays were used for the display for the left eye and the display for the right eye, it is also possible to use a single large display that has been divided into a region for the right eye and a region for the left eye.

### EXPLANATION OF REFERENCE NUMERALS

1 Visual field testing apparatus
10 HMD
11 Case
13 Display
14 Convex lens
15 Camera
16 Hot mirror
18 Emitter
30 Control apparatus
31 Line of sight detection unit
33 Target display unit
40 Visual recognition determination unit
41 Collision determination unit
50 Visual field testing unit
51 Central scotoma possessor testing unit
60 Arithmetic unit
65 Storage device
66 Line of sight log storage unit
67 Target storage unit
70 Communication cable

1. A visual field testing apparatus for testing a range of visual field of a user, the visual field testing apparatus comprising:
a display apparatus for displaying a target set consisting of members comprising a gazing target and a measurement target;
a line of sight detection unit that detects a line of sight of the user and that outputs line of sight information pertaining to a direction of the line of sight;
a visual recognition determination unit that carries out determination as to whether or not the user has visually recognized at least one of the members of the target set based on the line of sight information and position information of at least one of the members of the target set;
a target display unit that causes the target set to be displayed at the display apparatus; and
a visual field testing unit that carries out visual field testing by causing visual recognition determination to be carried out with respect to the measurement target by the visual recognition determination unit as the gazing target and the measurement target are sequentially displayed by the target display unit;
wherein the display apparatus has a display for a right eye and a display for a left eye; and
wherein the visual field testing unit has a central scotoma possessor testing mode in which the target display unit is controlled so as to cause the gazing target to be displayed by whichever of the display for the right eye or the display for the left eye is opposite an eye being tested.

2. The visual field testing apparatus according to claim 1 wherein, when in the central scotoma possessor testing mode, the visual field testing unit controls the target display unit so as to cause the gazing target to be displayed at the display for the right eye and so as to cause the measurement target to be displayed at the display for the left eye when testing the left eye of the user, and so as to cause the gazing target to be displayed at the display for the left eye and so as to cause the measurement target to be displayed at the display for the right eye when testing the right eye of the user.

3. The visual field testing apparatus according to claim 1 wherein, when in the central scotoma possessor testing mode, the visual field testing unit controls the target display unit so as to cause the gazing target to be displayed with increased brightness when the gazing target is displayed by whichever of the display for the right eye or the display for the left eye is opposite the eye being tested.

4. The visual field testing apparatus according to claim 1 wherein, when in the central scotoma possessor testing mode, the visual field testing unit controls the target display unit so as to cause the gazing target to be displayed so as to be of increased size when the gazing target is displayed by whichever of the display for the right eye or the display for the left eye is opposite the eye being tested.

5. The visual field testing apparatus according to claim 1 wherein the line of sight detection unit, the visual recognition determination unit, the target display unit, and the visual field testing unit employ a polar coordinate system.

6. The visual field testing apparatus according to claim 5 wherein the visual recognition determination unit determines that visual recognition has occurred when, in the polar coordinate system, a difference between a target angle of at least one of the members of the target set and a line of sight angle at the line of sight information is not greater than a prescribed angle.
